(19) United States
(12) Patent Application Publication
(10) Pub. No.: US 2007/0088030 A1
(43) Pub. Date: Apr. 19, 2007

(54) Aerosol formulations for the inhalation of beta-agonists

(76) Inventors: Barbara Niklaus-Humke, Damscheid (DE); Michael Nowak, Mainz (DE); Kirsten Radau, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/543,477

(22) Filed: Oct. 5, 2006

(30) Foreign Application Priority Data
Oct. 10, 2005 (EP) 05109376

(51) Int. Cl. A61K 31/538; A61K 31/4747; A61K 31/4745

(52) U.S. Cl. 514/230.5; 514/291; 514/278

(57) ABSTRACT

A pharmaceutical formulation, comprising:
(a) a compound of formula 1 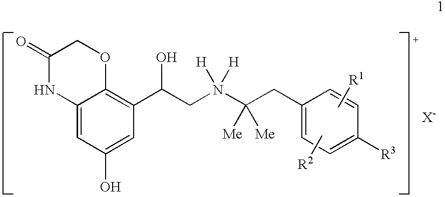 wherein:
   R¹ is hydrogen, C₁₋₄-alkyl, —O—C₁₋₄-alkyl, or halogen;
   R² is hydrogen, C₁₋₄-alkyl, —O—C₁₋₄-alkyl, or halogen;
   R³ is hydrogen, C₁₋₄-alkyl, —O—C₁₋₄-alkyl, halogen, OH, —O—C₁₋₄-alkylene-COOH, or —O— C₁₋₄-alkylene-COO—C₁₋₄-alkyl; and
   X⁻ denotes an anion with a single negative charge, or a tautomer, enantiomer, solvate, or hydrate thereof;
(b) a second active substance 2 selected from tiotropium salts, oxitropium salts, flutropium salts, ipratropium salts, glycopyrronium salts, and trospium salts, or a tautomer, enantiomer, solvate, or hydrate thereof; (c) at least one pharmacologically acceptable acid; and (d) a solvent selected from water, ethanol, or a mixture of water and ethanol.

### RELATED APPLICATIONS

This application claims priority to European Application No. 05109376.8, filed Oct. 10, 2005, which is hereby incorporated by reference.

### FIELD OF THE INVENTION

The present invention relates to a propellant-free aerosol formulation which contains one or more compounds of general formula 1 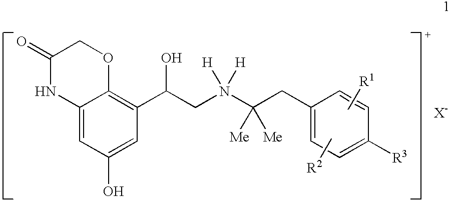
wherein the groups R¹, R², R³, and X⁻ may have the meanings given in the claims and in the description, and a further active substance 2, for inhalation.

### DETAILED DESCRIPTION OF THE INVENTION

The pharmaceutical formulations according to the invention are propellant-free pharmaceutical formulations, containing as active substance one or more compounds of general formula 1

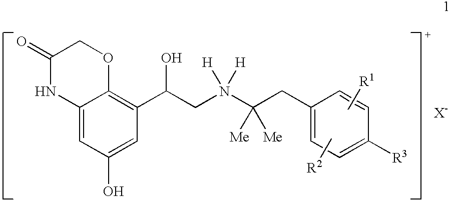

wherein:

R¹ denotes hydrogen, C₁₋₄-alkyl, —O—C₁₋₄-alkyl, or halogen;

R² denotes hydrogen, C₁₋₄-alkyl, —O—C₁₋₄-alkyl, or halogen;

R³ denotes hydrogen, C₁₋₄-alkyl, —O—C₁₋₄-alkyl, halogen, OH, —O—C₁₋₄-alkylene-COOH, or —O—C₁₋₄-alkylene-COO—C₁₋₄-alkyl; and

X⁻ denotes an anion with a single negative charge, preferably an anion with a single negative charge selected from among chloride, bromide, iodide, sulfate, phosphate, methanesulfonate, nitrate, maleate, acetate, benzoate, citrate, salicylate, trifluoroacetate, fumarate, tartrate, oxalate, succinate, benzoate, and p-toluenesulfonate,

optionally in the form of the tautomers, enantiomers, mixtures of the enantiomers, racemates, solvates, or hydrates thereof; a further active substance 2 selected from among the tiotropium salts, oxitropium salts, flutropium salts, ipratropium salts, glycopyrronium salts and trospium salts, optionally in the form of the tautomers, enantiomers, mixtures of the enantiomers, racemates, solvates, or hydrates thereof; at least one pharmacologically acceptable acid, optionally other pharmacologically acceptable excipients and/or complexing agents and, as solvent, water, ethanol, or a mixture of water and ethanol.

Preferred pharmaceutical formulations are those which contain the active substance 2 described above and compounds of general formula 1, wherein:

R¹ denotes hydrogen, methyl, ethyl, fluorine, or chlorine;

R² denotes hydrogen, methyl, ethyl, fluorine, or chlorine;

R³ denotes hydrogen, methyl, ethyl, propyl, OH, methoxy, ethoxy, fluorine, chlorine, bromine, —O—CH₂—COOH, —O—CH₂—COOmethyl, —O—CH₂—COOethyl, —O—CH₂—CH₂COOH, —O—CH₂—CH₂COOmethyl, —O—CH₂—CH₂COOethyl, —O—CH₂'CH₂—CH₂COOH, —O—CH₂—CH₂—CH₂COOmethyl, or —O—CH₂—CH₂—CH₂COOethyl; and

X⁻ denotes an anion with a single negative charge, preferably an anion with a single negative charge selected from among chloride, bromide, iodide, sulfate, phosphate, methanesulfonate, nitrate, maleate, acetate, benzoate, citrate, salicylate, trifluoroacetate, fumarate, tartrate, oxalate, succinate, benzoate, and p-toluenesulfonate,

optionally in the form of the tautomers, enantiomers, mixtures of the enantiomers, racemates, solvates, or hydrates thereof.

Preferred pharmaceutical formulations are those which contain the active substance 2 described above and compounds of general formula 1, wherein:

R¹ and R² each independently denote hydrogen or methyl, preferably hydrogen;

R³ denotes methyl, OH, methoxy, fluorine, chlorine, bromine, —O—CH₂—COOH, or —O—CH₂—COOethyl; and

X⁻ denotes an anion with a single negative charge selected from among the chloride, bromide, sulfate, methanesulfonate, maleate, acetate, benzoate, citrate, salicylate, trifluoroacetate, fumarate, tartrate, and succinate; optionally in the form of the tautomers, enantiomers, mixtures of the enantiomers, racemates, solvates, or hydrates thereof.

Also preferred are pharmaceutical formulations which contain the active substance 2 described above and compounds of general formula 1, wherein R³ denotes methoxy, ethoxy, fluorine, chlorine, bromine, —O—CH₂—COOH, —O—CH₂—COOmethyl, or —O—CH₂-COOethyl, and R¹, R, and X⁻ may have the meanings given above, optionally in the form of the tautomers, enantiomers, mixtures of the enantiomers, racemates, solvates, or hydrates thereof.

Also preferred are pharmaceutical formulations which contain the active substance 2 described above and compounds of general formula 1, wherein:

R¹ and R² each denote hydrogen;

R³ denotes OH, fluorine, chlorine, methoxy, ethoxy, or —O—CH₂—COOH, preferably OH, fluorine, chlorine, ethoxy, or methoxy; and

X⁻ may have one of the meanings given above,

optionally in the form of the tautomers, enantiomers, mixtures of the enantiomers, racemates, solvates, or hydrates thereof.

Also preferred are pharmaceutical formulations which contain the active substance 2 described above and the compounds of general formula 1 which are selected from among:
6-hydroxy-8-{1-hydroxy-2-[2-(4-methoxyphenyl)-1,1-dimethylethylamino]ethyl}-4H-benzo[1,4]oxazin-3-one;
6-hydroxy-8-{1-hydroxy-2-[2-(ethyl 4-phenoxyacetate)-1,1-dimethylethylamino]ethyl}4H-benzo[1,4]oxazin-3-one;
6-hydroxy-8-{1-hydroxy-2-[2-(4-phenoxyacetic acid)-1,1-dimethylethylamino]ethyl}-4H-benzo[1,4]oxazin-3-one;
8-{2-[1,1-dimethyl-2-(2,4,6-trimethylphenyl)ethylamino]-1-hydroxyethyl}-6-hydroxy-4H-benzo[1,4]oxazin-3-one;
6-hydroxy-8-{1-hydroxy-2-[2-(4-hydroxyphenyl)-1,1-dimethylethylamino]ethyl}4H-benzo[1,4]oxazin-3-one;
6-hydroxy-8-{1-hydroxy-2-[2-(4-isopropylphenyl)-1,1-dimethylethylamino]ethyl}-4H-benzo[1,4]oxazin-3-one;
8-{2-[2-(4-ethylphenyl)-1,1-dimethylethylamino]-1-hydroxyethyl}-6-hydroxy-4H-benzo[1,4]oxazin-3-one;
8-{2-[2-(4-fluoro-3-methylphenyl)-1,1-dimethylethylamino]-1-hydroxyethyl}-6-hydroxy-4H-benzo[1,4]oxazin-3-one;
8-{2-[2-(4-fluoro-2-methylphenyl)-1,1-dimethylethylamino]-1-hydroxyethyl}-6-hydroxy-4H-benzo[1,4]oxazin-3-one;
8-{2-[2-(2,4-difluorophenyl)-1,1-dimethylethylamino]-1-hydroxyethyl}-6-hydroxy-4H-benzo[1,4]oxazin-3-one;
8-{2-[2-(3,5-difluorophenyl)-1,1-dimethylethylamino]-1-hydroxyethyl}-6-hydroxy-4H-benzo[1,4]oxazin-3-one;
8-{2-[2-(4-ethoxyphenyl)-1,1-dimethylethylamino]-1-hydroxyethyl}-6-hydroxy4H-benzo[1,4]oxazin-3-one;
8-{2-[2-(3,5-dimethylphenyl)-1,1-dimethylethylamino]-1-hydroxyethyl}-6-hydroxy-4H-benzo[1,4]oxazin-3-one;
4-(4-{2-[2-hydroxy-2-(6-hydroxy-3-oxo-3,4-dihydro-2H-benzo[1,4]oxazin-8-yl)ethylamino]-2-methylpropyl}phenoxy)butyric acid;
8-{2-[2-(3,4-difluorophenyl)-1,1-dimethylethylamino]-1-hydroxyethyl}-6-hydroxy-4H-benzo[1,4]oxazin-3-one;
8-{2-[2-(2-chloro4-fluorophenyl)-1,1-dimethylethylamino]-1-hydroxyethyl}-6-hydroxy-4H-benzo[1,4]oxazin-3-one;
8-{2-[2-(4-chlorophenyl)-1,1-dimethylethylamino]-1-hydroxyethyl}-6-hydroxy4H-benzo[1,4]oxazin-3-one;
8-{2-[2-(4-bromophenyl)-1,1-dimethylethylamino]-1-hydroxyethyl}-6-hydroxy4H-benzo[1,4]oxazin-3-one;
8-{2-[2-(4-fluorophenyl)-1,1-dimethylethylamino]-1-hydroxyethyl}-6-hydroxy-4H-benzo[1,4]oxazin-3-one;
8-{2-[2-(4-fluoro-3-methoxyphenyl)-1,1-dimethylethylamino]-1-hydroxyethyl}-6-hydroxy-4H-benzo[1,4]oxazin-3-one;
8-{2-[2-(4-fluoro-2,6-dimethylphenyl)-1,1-dimethylethylamino]-1-hydroxyethyl}-6-hydroxy-4H-benzo [1,4]oxazin-3-one;
8-{2-[2-(4-chloro-2-methylphenyl)-1,1-dimethylethylamino]-1-hydroxyethyl}-6-hydroxy-4H-benzo[1,4]oxazin-3-one;
8-{2-[2-(4-chloro-3-fluorophenyl)-1,1-dimethylethylamino]-1-hydroxyethyl}-6-hydroxy-4H-benzo[1,4]oxazin-3-one;
8-{2-[2-(4-chloro-2-fluorophenyl)-1,1-dimethylethylamino]-1-hydroxyethyl}-6-hydroxy-4H-benzo[1,4]oxazin-3-one;
8-{2-[2-(3-chloro-4-fluorophenyl)-1,1-dimethylethylamino]-1-hydroxyethyl}-6-hydroxy-4H-benzo[1,4]oxazin-3-one;
8-{2-[2-(2,6-difluoro-4-methoxyphenyl)-1,1-dimethylethylamino]-1-hydroxyethyl}-6-hydroxy-4H-benzo[1,4]oxazin-3-one;
8-{2-[2-(2,5-difluoro-4-methoxyphenyl)-1,1-dimethylethylamino]-1-hydroxyethyl}-6-hydroxy-4H-benzo[1,4]oxazin-3-one;
8-1{2-[2-(4-fluoro-3,5-dimethylphenyl)-1,1-dimethylethylamino]-1-hydroxyethyl}-6-hydroxy-4H-benzo[1,4]oxazin-3-one;
8-{2-[2-(3,5-dichlorophenyl)-1,1-dimethylethylamino]-1-hydroxyethyl}-6-hydroxy-4H-benzo[1,4]oxazin-3-one;
8-{2-[2-(4-chloro-3-methylphenyl)-1,1-dimethylethylamino]-1-hydroxyethyl}-6-hydroxy-4H-benzo[1,4]oxazin-3-one;
8-{2-[2-(3,4,5-trifluorophenyl)-1,1-dimethylethylamino]-1-hydroxyethyl}-6-hydroxy-4H-benzo[1,4]oxazin-3-one;
8-{2-[2-(3-methylphenyl)-1,1-dimethylethylamino]- l-hydroxyethyl}-6-hydroxy4H-benzo[1,4]oxazin-3-one; and
8-{2-[2-(3,4-dichlorophenyl)-1,1-dimethylethylamino]-1-hydroxyethyl}-6-hydroxy-4H-benzo[1,4]oxazin-3-one,
   in each case in the form of an acid addition salt with an acid HX, wherein X⁻ may have one of the meanings given above, as well as optionally in the form of the tautomers, enantiomers, mixtures of the enantiomers, racemates, solvates, or hydrates thereof.

In the medicament combinations according to the invention, the active substance 2 is selected from among the anticholinergics consisting of tiotropium salts (2.1), oxitropium salts (2.2), flutropium salts (2.3), ipratropium salts (2.4), glycopyrronium salts (2.5), and trospium salts (2.6). The abovementioned anticholinergics may optionally have chiral carbon centers. In this case, the medicament combinations according to the invention may contain the anticholinergics in the form of their enantiomers, mixtures of enantiomers, or racemates, while preferably enantiomerically pure anticholinergics are used.

In the abovementioned salts 2.1 to 2.6 the cations tiotropium, oxitropium, flutropium, ipratropium, glycopyrronium, and trospium are the pharmacologically active constituents. An explicit reference to the abovementioned cations is indicated by the designations 2.1′ to 2.6′. Any reference to the abovementioned salts 2.1 to 2.6 naturally also includes a reference to the corresponding cations tiotropium (2.1′), oxitropium (2.2′), flutropium (2.3′), ipratropium (2.4′), glycopyrronium (2.5′), and trospium (2.6′).

By the salts 2.1 to 2.6 are meant according to the invention those compounds which contain, in addition to the cations tiotropium (2.1′), oxitropium (2.2′), flutropium (2.3′), ipratropium (2.4′), glycopyrronium (2.5′), and trospium (2.6′), chloride, bromide, iodide, sulfate, phosphate, methanesulfonate, nitrate, maleate, acetate, citrate, fumarate, tartrate, oxalate, succinate, benzoate, or p-toluenesulfonate as counter-ion (anion), the preferred counter-ions being chloride, bromide, iodide, sulfate, methanesulfonate, or p-toluenesulfonate. Of all the salts, the chlorides, bromides, iodides, and methanesulfonates are particularly preferred.

In the case of the trospium salts (2.6), the chloride is particularly preferred. In the case of the other salts 2.2 to 2.6, the methanesulfonates and bromides are of particular significance. Of particular importance are pharmaceutical combinations which contain tiotropium salts (2.1), oxitropium salts (2.2), or ipratropium salts (2.4), the associated bromides being of particular importance according to the invention. Tiotropium bromide (2.1) is of particular importance.

The abovementioned salts may optionally be present in the drug combinations according to the invention in the form of their solvates or hydrates, preferably in the form of their hydrates. In the case of tiotropium bromide, the drug combinations according to the invention preferably contain it in the form of the crystalline tiotropium bromide monohydrate, which is known from WO 02/30928, which is incorporated herein by reference. If tiotropium bromide is used in anhydrous form in the drug combinations according to the invention, anhydrous crystalline tiotropium bromide is preferably used, which is known from WO 03/000265, which is incorporated herein by reference.

Terms and Definitions Used

By the term “C₁₋₄-alkyl” (including those which are part of other groups) are meant branched and unbranched alkyl groups with 1 to 4 carbon atoms. Examples of these include: methyl, ethyl, n-propyl, isopropyl, n-butyl, isobutyl, sec-butyl, or tert-butyl. The abbreviations Me, Et, n-Pr, i-Pr, n-Bu, i-Bu, t-Bu, etc. may optionally also be used for the abovementioned groups. Unless stated otherwise, the definitions propyl and butyl include all the possible isomeric forms of the groups in question. Thus, for example, propyl includes n-propyl and isopropyl, butyl includes isobutyl, sec-butyl, and tert-butyl etc.

By the term “C₁₋₄-alkylene” (including those which are part of other groups) are meant branched and unbranched alkylene groups with 1 to 4 carbon atoms. Examples of these include: methylene, ethylene, propylene, 1-methylethylene, butylene, 1-methylpropylene, 1,1-dimethylethylene, or 1,2-dimethylethylene. Unless stated otherwise, the definitions propylene and butylene include all the possible isomeric forms of the groups in question with the same number of carbons. Thus, for example, propyl also includes 1-methylethylene and butylene includes 1-methylpropylene, 1,1-dimethylethylene, and 1,2-dimethylethylene.

Halogen within the scope of the present invention denotes fluorine, chlorine, bromine, or iodine. Unless stated otherwise, fluorine, chlorine and bromine are the preferred halogens.

By acid addition salts with pharmacologically acceptable acids are meant, for example, salts selected from the group comprising the hydrochloride, hydrobromide, hydriodide, hydrosulfate, hydrophosphate, hydromethanesulfonate, hydronitrate, hydromaleate, hydroacetate, hydrobenzoate, hydrocitrate, hydrofumarate, hydrotartrate, hydrooxalate, hydrosuccinate, hydrobenzoate, and hydro-p-toluenesulfonate, preferably the hydrochloride, hydrobromide, hydrosulfate, hydrophosphate, hydrofumarate, and hydromethanesulfonate. Of the abovementioned acid addition salts, the salts of hydrochloric acid, methanesulfonic acid, benzoic acid, and acetic acid are particularly preferred according to the invention.

The medicament formulations according to the invention contain as solvent pure water, pure ethanol or mixtures of ethanol and water. If ethanol-water mixtures are used, the percentage by mass of ethanol in these mixtures is preferably in the range between 5% and 99% ethanol, particularly preferably in the range from 10% to 96% ethanol. Most particularly preferred medicament formulations for the purposes of the present invention contain as solvent pure water, pure ethanol or ethanol-water mixtures containing between 50% and 92%, particularly preferably between 69% and 91% ethanol. If desired, other co-solvents may be used in addition to ethanol and water. Preferably, however, no other solvent is used according to the invention.

The compounds according to the invention may be prepared analogously to methods already known from the prior art. Suitable methods of preparation are known, for example, from U.S. Pat. No. 4,460,581, which is incorporated herein by reference.

The compounds of formula 1 may optionally be present in the medicament formulations according to the invention in the form of their tautomers. By tautomerism is meant the occurrence of isomeric compounds which are formed by the shifting of σ or π bonds and may be present in equilibrium. Examples of possible tautomeric forms of the compounds of formula 1 are

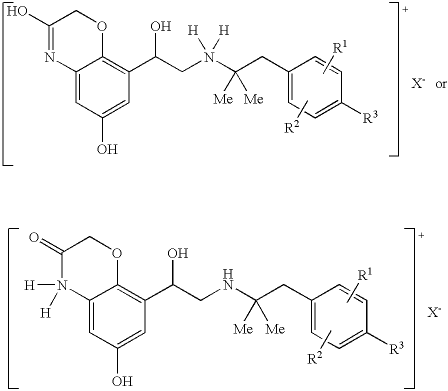

In another aspect, the present invention relates to pharmaceutical formulations which contain the abovementioned compounds of formula 1 in the form of the individual optical isomers, mixtures of individual enantiomers or racemates. Particularly preferred are medicament formulations which contain the abovementioned compounds of formula 1 in the form of the enantiomerically pure compounds, while the R-enantiomers of the compounds of formula 1 according to the invention are of particular importance. These R-enantiomers may be represented by general formula R-1 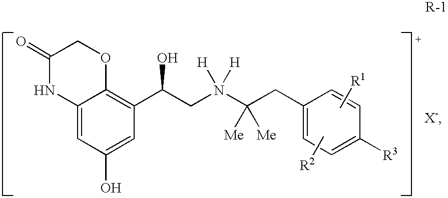
wherein the groups R¹, R², R³, and X⁻ may have the meanings given above.

Within the scope of the present invention it is particularly preferable to use those compounds of formula 1 wherein X⁻ is selected from among the chloride, maleate, salicylate, fumarate or succinate, optionally in the form of the hydrates or solvates thereof. Particularly preferred according to the invention are those formulations which contain the compound of formula 1, wherein X⁻ denotes chloride.

References to the compound of formula 1 always include within the scope of the present invention all possible amorphous and crystalline modifications of this compound. References to the compound of formula 1 also include within the scope of the present invention all the possible solvates and hydrates which may be formed from this compound.

Any reference to the compound 1′ within the scope of the present invention is to be regarded as a reference to the pharmacologically active free base of the following formula 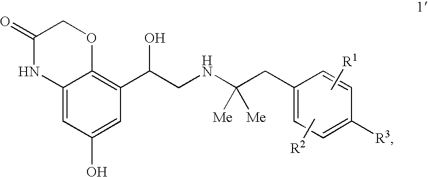
contained in the salts 1 wherein the groups R¹, R², R³, and X⁻ may have the meanings given above.

In another aspect, the present invention relates to medicament formulations containing an active substance 2 and a free base of formula 1′, wherein the groups R¹, R², R³, and X⁻ may have the meanings given above, optionally in the form of their tautomers, enantiomers, mixtures of enantiomers, racemates, solvates, or hydrates, at least one pharmacologically acceptable acid, optionally other pharmacologically acceptable excipients and/or complexing agents and, as solvent, water, ethanol, or a mixture of water and ethanol.

In another aspect, the present invention relates to the use of the pharmaceutical formulations according to the invention for preparing a pharmaceutical composition for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome), and all forms of pulmonary edema.

Preferably the medicament formulations according to the invention are used as specified above for preparing a pharmaceutical composition for the treatment of obstructive pulmonary diseases selected from among bronchial asthma, pediatric asthma, severe asthma, acute asthma attacks, chronic bronchitis, and chronic obstructive pulmonary disease (COPD), while it is particularly preferable according to the invention to use them for preparing a pharmaceutical composition for the treatment of bronchial asthma or COPD.

It is also preferable to use the medicament formulations according to the invention for preparing a pharmaceutical composition for the treatment of pulmonary emphysema which has its origins in COPD or α1-proteinase inhibitor deficiency.

It is also preferable to use the medicament formulations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumors, such as, for example, lymphangiosis carcinomatosa, bronchoalveolar carcinoma, and lymphomas.

It is also preferable to use the medicament formulations according to the invention for preparing a pharmaceutical composition for the treatment of interstitial pulmonary diseases selected from among pneumonia caused by infections, such as, for example, infection by viruses, bacteria, fungi, protozoa, helminths or other pathogens, pneumonitis caused by various factors, such as, for example, aspiration and left heart insufficiency, radiation-induced pneumonitis or fibrosis, collagenoses, such as, for example, lupus erythematodes, systemic scleroderma or sarcoidosis, granulomatoses, such as, for example, Boeck's disease, idiopathic interstitial pneumonia, or idiopathic pulmonary fibrosis (IPF).

It is also preferable to use the medicament formulations according to the invention for preparing a pharmaceutical composition for the treatment of cystic fibrosis or mucoviscidosis.

It is also preferable to use the medicament formulations according to the invention for preparing a pharmaceutical composition for the treatment of bronchitis, such as, for example, bronchitis caused by bacterial or viral infection, allergic bronchitis, and toxic bronchitis.

It is also preferable to use the medicament formulations according to the invention for preparing a pharmaceutical composition for the treatment of bronchiectasis.

It is also preferable to use the medicament formulations according to the invention for preparing a pharmaceutical composition for the treatment of ARDS (adult respiratory distress syndrome).

It is also preferable to use the medicament formulations according to the invention for preparing a pharmaceutical composition for the treatment of pulmonary edema, for example, toxic pulmonary edema after aspiration or inhalation of toxic substances and foreign substances.

Most preferably, the present invention relates to the use of the pharmaceutical formulations according to the invention for preparing a pharmaceutical composition for the treatment of asthma or COPD. Also of particular importance is the abovementioned use for preparing a pharmaceutical composition for once-a-day treatment of inflammatory and obstructive respiratory complaints, particularly for the once-a-day treatment of asthma or COPD.

Moreover the present invention relates to a process for the treatment of the abovementioned diseases, characterized in that one or more of the abovementioned medicament formulations according to the invention are administered in therapeutically effective amounts.

The present invention relates to liquid active substance formulations of these compounds which can be administered by inhalation; the liquid formulations according to the invention have to meet high quality standards. The formulations according to the invention may be inhaled by oral or nasal route. To achieve an optimum distribution of the active substances in the lung, it makes sense to use a liquid formulation without propellant gases administered using suitable inhalers. A formulation of this kind may be inhaled both by oral route and by nasal route. Those inhalers which are capable of nebulizing a small amount of a liquid formulation in the dosage needed for therapeutic purposes within a few seconds into an aerosol suitable for therapeutic inhalation are particularly suitable. Within the scope of the invention, preferred nebulizers are those in which an amount of less than 100 microliters, preferably less than 50 microliters, most preferably less than 25 microliters of active substance solution can be nebulized preferably in one puff or two puffs to form an aerosol having an average particle size of less than 20 microns, preferably less than 10 microns, so that the inhalable part of the aerosol already corresponds to the therapeutically effective quantity.

An apparatus of this kind for propellant-free delivery of a metered quantity of a liquid pharmaceutical composition for inhalation is described for example in International Patent Application WO 91/14468 (corresponding to U.S. Pat. No. 5,497,944, which is hereby incorporated by reference) and also in WO 97/12687 (corresponding to U.S. Pat. No. 5,964,416, which is hereby incorporated by reference) (cf in particular FIGS. 6a and 6b). The nebulizers (devices) described therein are also known by the name RESPIMATS. In a nebulizer of this kind a pharmaceutical solution is converted by means of a high pressure of up to 500 bar into an aerosol destined for the lungs, which is sprayed.

In inhalers of this kind, the formulations of solutions are stored in a reservoir. It is essential that the active substance formulations used are sufficiently stable when stored and at the same time are such that they can be administered directly, if possible without any further handling, in accordance with their medical purpose. Moreover, they must not contain any ingredients which might interact with the inhaler in such a way as to damage the inhaler or the pharmaceutical quality of the solution or of the aerosol produced.

To nebulize the solution, a special nozzle is used as described for example in WO 94/07607 (corresponding to U.S. Pat. Nos. 5,911,851; 6,007,676; and 6,503,362, each of which is hereby incorporated by reference) or WO 99/16530 (corresponding to U.S. Patent Application Pub. No. 2004/0159319, which is hereby incorporated by reference). Reference is expressly made here to both these publications.

The aim of the invention is to provide an aqueous, ethanolic, or aqueous-ethanolic formulation of the compound of formula 1, optionally in the form of the tautomers, enantiomers, mixtures of the enantiomers, racemates, solvates, or hydrates thereof; a further active substance 2 selected from among the tiotropium salts, oxitropium salts, flutropium salts, ipratropium salts, glycopyrronium salts, and trospium salts, optionally in the form of the tautomers, enantiomers, mixtures of the enantiomers, racemates, solvates, or hydrates thereof, which meets the high standards required to ensure optimum nebulization of a solution using the inhalers mentioned above. The active substance formulations according to the invention must be of sufficiently high pharmaceutical quality, i.e., they should be pharmaceutically stable over a storage time of some years, preferably at least one year, more preferably two years.

These propellant-free formulations of solutions must also be capable of being nebulized by means of an inhaler under pressure, while the composition delivered in the aerosol produced is within a specified range.

According to the invention the formulation preferably contains the active substance 2 and only one compound of formula 1. However, the formulation may also contain a mixture of different salts of formula 1. If the medicament formulations according to the invention contain different salts of formula 1, the preferred formulations according to the invention are those wherein the various salts denote different salts of the same free base of formula 1′.

The concentration of the compound of formula 1 based on the proportion of pharmacologically active free base 1′ in the pharmaceutical preparation according to the invention is about 0.1 to 2000 mg per 100 mL, according to the invention, preferably about 0.5 to 1100 mg per 100 mL, particularly preferably 0.75 to 500 mg per 100 mL. Particularly preferably, 100 mL of the formulations according to the invention contain about 1 to about 250 mg of 1′.

The concentration of the compound of formula 2 based on the proportion of pharmacologically active free cation of the salt 2.1 in the pharmaceutical preparation according to the invention is about 0.1 to 2000 mg per 100 mL according to the invention, preferably about 0.5 to 1100 mg per 100 mL, particularly preferably 0.75 to 500 mg per 100 mL. Particularly preferably 100 mL of the formulations according to the invention contain about 1 to about 250 mg of the free cation of the salt 2.1.

The pH of the formulation according to the invention is preferably in a range from 2.0 to 6.5, preferably between 2.5 and 3.5, particularly preferably between about 2.7 and 3.1 in purely aqueous solutions.

If the compounds 1 and 2 are dissolved in ethanol or in mixtures of ethanol and water, the pH of the formulation according to the invention is preferably in the range from 2.0 to 6.5, preferably between 2.5 and 5.5, particularly preferably between about 2.7 and 5.0.

The pH is adjusted by the addition of pharmacologically acceptable acids. Pharmacologically acceptable inorganic acids or organic acids may be used for this purpose. Examples of preferred inorganic acids are selected from the group consisting of hydrochloric acid, hydrobromic acid, nitric acid, sulfuric acid, and phosphoric acid.

Examples of particularly suitable organic acids are selected from the group consisting of ascorbic acid, citric acid, malic acid, tartaric acid, maleic acid, succinic acid, fumaric acid, acetic acid, fonnic acid, propionic acid, sorbic acid, benzoic acid, methanesulfonic acid, and benzenesulfonic acid. Preferred inorganic acids are hydrochloric acid, phosphoric acid, and sulfuric acid, of which hydrochloric acid is particularly preferred according to the invention. Of the organic acids, ascorbic acid, fumaric acid, methanesulfonic acid, and citric acid are preferred. If desired, mixtures of the abovementioned acids may also be used, particularly in the case of acids which have other properties in addition to their acidifying properties, e.g., those which act as flavorings or antioxidants, such as, for example, citric acid or ascorbic acid. If desired, pharmacologically acceptable bases may also be used to titrate the pH precisely. Suitable bases include, for example, alkali metal hydroxides and alkali metal carbonates. The preferred alkali metal ion is sodium. If bases of this kind are used, care must be taken to ensure that the resulting salts, which are then contained in the finished pharmaceutical formulation, are pharmacologically compatible with the abovementioned acid.

The formulations according to the invention may contain complexing agents as other ingredients. By complexing agents are meant within the scope of the present invention molecules which are capable of entering into complex bonds. Preferably, these compounds should have the effect of complexing cations, most preferably metal cations. The formulations according to the invention preferably contain edetic acid (EDTA) or one of the known salts thereof, e.g., sodium EDTA or disodium EDTA, as complexing agent. Preferably, sodium edetate is used, optionally in the form of its hydrates, more preferably in the form of its dihydrate. If complexing agents are used within the formulations according to the invention, their content is preferably in the range from 0.1 to 50 mg per 100 mL, more preferably in the range from 0.5 to 25 mg per 100 mL of the formulation according to the invention. Preferably, the formulations according to the invention contain a complexing agent in an amount of about 0.75 to 15 mg per 100 mL, more preferably about 1 to 12 mg per 100 mL of the formulation according to the invention.

The remarks made concerning disodium edetate also apply analogously to other possible additives which are comparable to EDTA or the salts thereof, which have complexing properties and can be used instead of them, such as for example nitrilotriacetic acid and the salts thereof.

Other pharmacologically acceptable excipients may also be added to the formulation according to the invention. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilizers, antioxidants, and/or preservatives which prolong the shelf life of the finished pharmaceutical formulation, as well as flavorings, vitamins and/or other additives known in the art. The additives also include pharmacologically acceptable salts such as sodium chloride, for example.

The preferred excipients include antioxidants such as ascorbic acid, for example, provided that it has not already been used to adjust the pH, propylgallate, BHA (butylhydroxyanisol), BHT (butylhydroxytoluene), TBHQ (tert-butylhydroxyquinone), vitamin A, vitamin E, tocopherols, and similar vitamins or provitamins occurring in the human body.

Preservatives can be added to protect the formulation from contamination with pathogenic bacteria. Suitable preservatives are those known from the prior art, particularly benzalkonium chloride or benzoic acid or benzoates such as sodium benzoate in the concentration known from the prior art. Preferably, benzalkonium chloride is added to the formulation according to the invention. The amount of benzalkonium chloride is between 1 mg and 50 mg per 100 mL of formulation, preferably about 2 to 15 mg per 100 mL, more preferably about 3 to 12 mg per 100 mL of the formulation according to the invention, most preferably about 4 to 11 mg per 100 mL of the formulation according to the invention. Benzalkonium chloride may also be used according to the invention in admixture with other preservatives.

Preferred formulations contain only benzalkonium chloride, sodium edetate and the acid needed to adjust the pH, in addition to the solvent water, the compounds of formula 1 and active substance 2.

The pharmaceutical formulations according to the invention are preferably used in an inhaler of the kind described hereinbefore in order to produce the propellant-free aerosols according to the invention. At this point we should once again expressly mention the patent documents described hereinbefore, to which reference is hereby made. As described at the beginning, a further developed embodiment of the preferred inhaler is disclosed in WO 97/12687 (cf. in particular FIGS. 6a and 6b and the associated passages of description). This nebulizer (RESPIMAT®) can advantageously be used to produce the inhalable aerosols according to the invention. Because of its cylindrical shape and handy size of less than 9 to 15 cm long and 2 to 4 cm wide, the device can be carried anywhere by the patient. The nebulizer sprays a defined volume of the pharmaceutical formulation out through small nozzles at high pressures, so as to produce inhalable aerosols. The preferred atomizer essentially consists of an upper housing part, a pump housing, a nozzle, a locking clamp, a spring housing, a spring and a storage container, characterized by:
a pump housing fixed in the upper housing part and carrying at one end a nozzle body with the nozzle or nozzle arrangement,
a hollow piston with valve body,
a power take-off flange in which the hollow body is fixed and which is located in the upper housing part,
a locking clamping mechanism located in the upper housing part,
a spring housing with the spring located therein, which is rotatably mounted on the upper housing part by means of a rotary bearing, and
a lower housing part which is fitted onto the spring housing in the axial direction.

The hollow piston with valve body corresponds to a device disclosed in WO 97/12687. It projects partially into the cylinder of the pump housing and is disposed to be axially movable in the cylinder. Reference is made particularly to FIGS. 1-4, especially FIG. 3, and the associated passages of description in the abovementioned International Patent Application. At the moment of release of the spring the hollow piston with valve body exerts, at its high pressure end, a pressure of 5 to 60 MPa (about 50 to 600 bar), preferably 10 to 60 MPa (about 100 to 600 bar) on the fluid, the measured amount of active substance solution. Volumes of 10 to 50 microliters are preferred, volumes of 10 to 20 microliters are more preferable, whilst a volume of 10 to 15 microliters per actuation is particularly to preferred.

The valve body is preferably mounted at the end of the hollow piston which faces the nozzle body.

The nozzle in the nozzle body is preferably microstructured, i.e., produced by micro-engineering. Microstructured nozzle bodies are disclosed, for example, in WO 99/16530; reference is hereby made to the contents of this specification, especially FIG. 1 and the associated description. The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns and the length being 7 to 9 microns.

If there is a plurality of nozzle openings, preferably two, the directions of spraying of the nozzles in the nozzle body may run parallel to each other or may be inclined relative to one another in the direction of the nozzle opening. In the case of a nozzle body having at least two nozzle openings at the outlet end, the directions of spraying may be inclined relative to one another at an angle of 20 degrees to 160 degrees, preferably at an angle of 60 to 150 degrees, most preferably 80 to 100°. The nozzle openings are preferably arranged at a spacing of 10 to 200 microns, more preferably at a spacing of 10 to 100 microns, still more preferably 30 to 70 microns. A spacing of 50 microns is most preferred. The directions of spraying therefore meet in the region of the nozzle openings.

As already mentioned, the liquid pharmaceutical preparation hits the nozzle body at an entry pressure of up to 600 bar, preferably 200 to 300 bar and is atomized through the nozzle openings into an inhalable aerosol. The preferred particle sizes of the aerosol are up to 20 microns, preferably 3 to 10 microns.

The locking clamping mechanism contains a spring, preferably a cylindrical helical compression spring, as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g. a helical sliding gear, by an external torque which is generated when the upper housing part is turned relative to the spring housing in the lower housing part. In this case, the upper housing part and the power take-off flange contain a single- or multi-speed spline gear.

The locking member with the engaging locking surfaces is arranged in an annular configuration around the power take-off flange. It consists for example of a ring of plastics or metal which is inherently radially elastically deformable. The ring is arranged in a plane perpendicular to the axis of the atomizer. After the locking of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomizer, and the deformable ring is thereby deformed in the annular plane. Details of the construction of the locking clamping mechanism are described in WO 97/20590 (corresponding to U.S. Pat. No. 6,453,795, which is hereby incorporated by reference).

The lower housing part is pushed axially over the spring housing and covers the bearing, the drive for the spindle and the storage container for the fluid.

When the atomizer is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360°, e.g., 180°. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle.

If desired, a plurality of replaceable storage containers containing the fluid to be atomized can be inserted in the atomizer one after another and then used. The storage container contains the aqueous aerosol preparation according to the invention.

The atomizing process is initiated by gently pressing the actuating button. The clamping mechanism then opens the way for the power take-off component. The biased spring pushes the piston into the cylinder in the pump housing. The fluid emerges from the nozzle of the atomizer in the form of a spray.

Further details of the construction are disclosed in PCT applications WO 97/12683 and WO 97/20590, to which reference is hereby made.

The components of the atomizer (nebulizer) are made of a material suitable for their function. The housing of the atomizer and, if the function allows, other parts as well are preferably made of plastics, e.g., by injection molding. For medical applications, physiologically acceptable materials are used.

FIGS. 6a/b of WO 97/12687 show the RESPIMAT® nebulizer with which the aqueous aerosol preparations according to the invention can advantageously be inhaled. FIG. 6a shows a longitudinal section through the atomizer with the spring under tension, FIG. 6b shows a longitudinal section through the atomizer with the spring released.

The upper housing part (51) contains the pump housing (52), on the end of which is mounted the holder (53) for the atomizer nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66). The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomized. The storage container is closed off by the stopper (73), through which the hollow piston projects into the storage container and dips its end into the fluid (supply of active substance solution).

The spindle (74) for the mechanical counter is mounted on the outside of the spring housing. The drive pinion (75) is located at the end of the spindle facing the upper housing part. On the spindle is the slider (76).

The nebulizer described above is suitable for nebulizing the aerosol preparations according to the invention to form an aerosol suitable for inhalation.

If the formulation according to the invention is nebulized using the method described above (RESPIMAT®), the mass expelled, in at least 97%, preferably at least 98% of all the actuations of the inhaler (puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

However, the formulation according to the invention can also be nebulized using inhalers other than those described above, for example jet-stream inhalers.

The present invention also relates to an inhalation kit consisting of one of the pharmaceutical preparations according to the invention described above and an inhaler suitable for nebulizing this pharmaceutical preparation. The present invention preferably relates to an inhalation kit consisting of one of the pharmaceutical preparations according to the invention described above and the RESPIMAT® inhaler described above.

If the formulation is to be administered nasally using the RESPIMAT® device described above, this atomizer can be provided with an attachment on the mouthpiece which is designed in the manner of a cylindrical pyramid, i.e., a pyramid with a round or oval cross-section or a tapering, round or oval cylinder. This attachment is hollow on the inside and has two openings. One of the openings may be fitted over the mouthpiece and the other opening at the pointed end can be inserted in a nostril.

Thus, this attachment is preferably in the form of the spout of a conventional nasal spray. The attachment may be constructed so as to be detachably or non-detachably connected to the mouthpiece. An attachment of this kind may also replace the mouthpiece.

The examples of formulations given below serve as illustrations without restricting the subject matter of the present invention to the compositions shown by way of example.

### EXAMPLES

As already mentioned, the compounds of formula 1 may be prepared in a manner known per se. Compounds mentioned by way of example and preferred within the scope of the invention are listed below. Thus, preferred pharmaceutical formulations are those which contain an active substance 2 and compounds of general formula 1, selected from among the following:

### Example 1

### 6-hydroxy-8-{1-hydroxy-2-[2-(4-hydroxy-2,6-dimethylphenyl)-1,1-dimethylethylamino]ethyl}4H-benzo[1,4]oxazin-3-one methanesulfonate;

### Example 2

### 8-{2-[2-(4-fluorophenyl)-1,1-dimethylethylamino]-1-hydroxyethyl}-6-hydroxy-4H-benzo[1,4]oxazin-3-one acid addition salt;

### Example 3

### 6-hydroxy-8-{1-hydroxy-2-[2-(4-methoxyphenyl)-1,1-dimethylethylamino]ethyl}4H-benzo[1,4]oxazin-3-one hydrochloride;

### Example 4

### 6-hydroxy-8-{1-hydroxy-2-[2-(ethyl 4-phenoxyacetate)-1,1-dimethylethylamino]ethyl}4H-benzo[1,4]oxazin-3-one hydrochloride;

### Example 5

### 6-hydroxy-8-{1-hydroxy-2-[2-(4-phenoxyacetic acid)-1,1-dimethylethylamino]ethyl}4H-benzo[1,4]oxazin-3-one hydrochloride;

### Example 6

### 8-{2-[1,1-dimethyl-2-(2,4,6-trimethylphenyl)ethylamino] 1-hydroxyethyl}-6-hydroxy4H-benzo[1,4]oxazin-3-one hydrochloride;

### Example 7

### 6-hydroxy-8-{1-hydroxy-2-[2-(4-hydroxyphenyl)-1,1-dimethylethylamino]ethyl}4H-benzo[1,4]oxazin-3-one hydrochloride;

### Example 8

### 6-hydroxy-8-{1-hydroxy-2-[2-(4-isopropylphenyl)-1,1-dimethylethylamino]ethyl}4H-benzo[1,4]oxazin-3-one hydrochloride;

### Example 9

### 8-{2-[2-(4-ethylphenyl)-1,1-dimethylethylamino]-1-hydroxyethyl}-6-hydroxy-4H-benzo[1,4]oxazin-3-one hydrochloride;

### Example 10

### 8-{2-[2-(4-fluoro-3-methylphenyl)-1,1-dimethylethylamino]-1-hydroxyethyl}-6-hydroxy4H-benzo[1,4]oxazin-3-one hydrochloride;

### Example 11

### 8-{2-[2-(4-fluoro-2-methylphenyl)-1,1-dimethylethylamino]-1-hydroxyethyl}-6-hydroxy4H-benzo[1,4]oxazin-3-one hydrochloride;

### Example 12

### 8-{2-[2-(2,4-difluorophenyl)-1,1-dimethylethylamino]-1-hydroxyethyl}-6-hydroxy-4H-benzo[1,4]oxazin-3-one hydrochloride;

### Example 13

### 8-{2-[2-(3,5-difluorophenyl)-1,1-dimethylethylamino]-1-hydroxyethyl}-6-hydroxy-4H-benzo[1,4]oxazin-3-one hydrochloride;

### Example 14

### 8-{2-[2-(4-ethoxyphenyl)-1,1-dimethylethylamino]-1-hydroxyethyl}-6-hydroxy-4H-benzo[1,4]oxazin-3-one hydrochloride;

### Example 15

### 8-{2-[2-(3,5-dimethylphenyl)-1,1-dimethylethylamino]-1-hydroxyethyl}-6-hydroxy-4H-benzo[1,4]oxazin-3-one hydrochloride;

### Example 16

### 4-{4-2-[2-hydroxy-2-(6-hydroxy-3-oxo-3,4-dihydro-2H-benzo[1,4]oxazin-8-yl)ethylamino]-2-methylpropyl}phenoxy)butyric acid-acid addition salt;

### Example 17

### 8-{2-[2-(3,4-difluorophenyl)-1,1-dimethylethylamino]-1-hydroxyethyl}-6-hydroxy-4H-benzo[1,4]oxazin-3-one trifluoroacetate;

### Example 18

### 8-{2-[2-(2-chloro-4-fluorophenyl)-1,1-dimethylethylamino]-1-hydroxyethyl}-6-hydroxy4H-benzo[1,4]oxazin-3-one trifluoroacetate;

### Example 19

### 8-{2-[2-(4-chlorophenyl)-1,1-dimethylethylamino]-1-hydroxyethyl}-6-hydroxy-4H-benzo[1,4]oxazin-3-one acid addition salt;

### Example 20

### 8-{2-[2-(4-bromophenyl)-1,1-dimethylethylamino]-1-hydroxyethyl}-6-hydroxy-4H-benzo[1,4]oxazin-3-one acid addition salt;

### Example 21

### 8-{2-[2-(3-methylphenyl)-1,1-dimethylethylamino]-1-hydroxyethyl}-6-hydroxy-4H-benzo[1,4]oxazin-3-one acid addition salt;

### Example 22

### 8-{2-[2-(4-fluoro-3-methoxyphenyl)-1,1-dimethylethylamino]-1-hydroxyethyl}-6-hydroxy4H-benzo[1,4]oxazin-3-one acid addition salt;

### Example 23

### 8-{2-[2-(4-fluoro-2,6-dimethylphenyl)-1,1-dimethylethylamino]-1-hydroxyethyl}-6-hydroxy4H-benzo[1,4]oxazin-3-one acid addition salt;

### Example 24

### 8-{2-[2-(4-chloro-2-methylphenyl)-1,1-dimethylethylamino]-1-hydroxyethyl}-6-hydroxy4H-benzo[1,4]oxazin-3-one acid addition salt;

### Example 25

### 8-{2-[2-(4-chloro-3-fluorophenyl)-1,1-dimethylethylamino]-1-hydroxyethyl}-6-hydroxy4H-benzo[1,4]oxazin-3-one acid addition salt;

### Example 26

### 8-{2-[2-(4-chloro-2-fluorophenyl)-1,1-dimethylethylamino]-1-hydroxyethyl}-6-hydroxy4H-benzo[1,4]oxazin-3-one acid addition salt;

### Example 27

### 8-{2-[2-(3-chloro-4-fluorophenyl)-1,1-dimethylethylamino]-1-hydroxyethyl}-6-hydroxy4H-benzo[1,4]oxazin-3-one acid addition salt;

### Example 28

### 8-{2-[2-(2,6-difluoro-4-methoxyphenyl)-1,1-dimethylethylamino]-1-hydroxyethyl}-6-hydroxy-4H-benzo[1,4]oxazin-3-one acid addition salt;

### Example 29

### 8-{2-[2-(2.5-difluoro-4-methoxyphenyl)-1,1-dimethylethylamino]-1-hydroxyethyl}-6-hydroxy4H-benzo[1,4]oxazin-3-one acid addition salt;

### Example 30

### 8-{2-[2-(4-fluoro-3,5-dimethylphenyl)-1,1-dimethylethylamino]-1-hydroxyethyl}-6-hydroxy4H-benzo[1,4]oxazin-3-one acid addition salt;

### Example 31

### 8-{2-[2-(3,5-dichlorophenyl)-1,1-dimethylethylamino]-1-hydroxyethyl}-6-hydroxy-4H-benzo[1,4]oxazin-3-one acid addition salt;

### Example 32

### 8-{2-[2-(4-chloro-3-methylphenyl)-1,1-dimethylethylamino]-1-hydroxyethyl}-6-hydroxy4H-benzo[1,4]oxazin-3-one acid addition salt;

### Example 33

### 8-{2-[2-(3,4,5-trifluorophenyl)-1,1-dimethylethylamino]-1-hydroxyethyl}-6-hydroxy-4H-benzo[1,4]oxazin-3-one acid addition salt; and

### Example 34

### 8-{2-[2-(3,4-dichlorophenyl)-1,1-dimethylethylamino]-1,1-hydroxyethyl}-6-hydroxy-4H-benzo[1,4]oxazin-3-one acid addition salt,

optionally in the form of an acid addition salt with an acid HX, wherein X⁻ may have one of the meanings given above, as well as optionally in the form of the tautomers, enantiomers, mixtures of the enantiomers, racemates, solvates, or hydrates thereof.

The Table that follows is a compilation of formulation examples according to the invention. The abbreviation BA-Cl denotes benzalkonium chloride and EDTA represents disodium edetate dihydrate.

The active substances 1 and 2.1 mentioned are used in the form of salts and/or hydrates, but are specified here based on the mass of the free base of 1 and of the free cation of 2.1. The compound 1 is used in the following Examples in the form of the hydrochloride, hydrotetrafluoroacetate or hydromethanesulfonate, while compound 2 is used as a monohydrate of the bromide.

A) The Table that follows is a compilation of formulation examples according to the invention of the R-enantiomer of the compound of Example 1 and the active substance 2.1 in the form of base and cation. 100 mL of medicament formulation contains the following, in purified water or water for injections:

| | 1′ (base) | 2.1′ (cation) | BA-Cl | EDTA | pH |
|---|---|---|---|---|---|
| Example | (mg) | (mg) | (mg) | (mg) | (HCl) |
| 1. | 1000 | 1000 | — | 2 | 2.9 |
| 2. | 500 | 500 | 10 | 10 | 2.7 |
| 3. | 500 | 250 | 4 | 10 | 2.9 |
| 4. | 500 | 250 | 5 | 10 | 2.9 |
| 5. | 500 | 85 | 10 | 11 | 2.9 |
| 6. | 250 | 500 | 10 | 9 | 3.1 |
| 7. | 85 | 500 | 8 | 10 | 2.9 |
| 8. | 200 | 45 | 15 | — | 2.9 |
| 9. | 200 | 23 | 11 | 12 | 2.7 |
| 10. | 200 | 23 | 10 | 10 | 2.7 |
| 11. | 90 | 23 | 10 | 10 | 2.8 |
| 12. | 90 | 23 | 10 | 5 | 3.1 |
| 13. | 45 | 23 | 10 | 10 | 2.9 |
| 14. | 45 | 23 | 10 | 5 | 3.0 |
| 15. | 23 | 23 | 10 | 10 | 2.9 |
| 16. | 23 | 23 | 10 | 5 | 2.9 |
| 17. | 23 | 45 | 11 | 25 | 3.0 |
| 18. | 23 | 45 | 10 | 10 | 3.0 |
| 19. | 9 | 23 | 10 | 10 | 3.1 |
| 20. | 11 | 23 | 10 | 25 | 3.1 |
| 21. | 9 | 23 | 10 | 5 | 2.8 |
| 22. | 5 | 23 | 10 | 10 | 3.1 |
| 23. | 5 | 23 | 10 | 50 | 3.1 |
| 24. | 5 | 23 | 10 | 5 | 2.7 |
| 25. | 5 | 45 | 12 | 5 | 3.0 |
| 26. | 1 | 1 | 10 | 7 | 3.5 |
| 27. | 1 | 1 | 10 | 7 | 3.0 |
| 28. | 0.1 | 0.1 | 10 | 15 | 3.5 |
| 29. | 0.1 | 0.1 | 10 | 15 | 3.0 |

B) The Table that follows is a compilation of formulation examples according to the invention of the R-enantiomer of the compound of Example 3 and the active substance 2.1 in the form of base and cation. 100 mL of medicament formulation contains the following, in purified water or water for injections:

| | 1′ (base) | 2.1′ (cation) | BA-Cl | EDTA | pH |
|---|---|---|---|---|---|
| Example | (mg) | (mg) | (mg) | (mg) | (HCl) |
| 1. | 1000 | 1000 | — | 2 | 2.9 |
| 2. | 500 | 500 | 10 | 10 | 2.7 |
| 3. | 500 | 250 | 4 | 10 | 2.9 |
| 4. | 500 | 250 | 5 | 10 | 2.9 |
| 5. | 500 | 85 | 10 | 11 | 2.9 |
| 6. | 250 | 500 | 10 | 9 | 3.1 |
| 7. | 85 | 500 | 8 | 10 | 2.9 |
| 8. | 200 | 45 | 15 | — | 2.9 |
| 9. | 200 | 23 | 11 | 12 | 2.7 |
| 10. | 200 | 23 | 10 | 10 | 2.7 |
| 11. | 90 | 23 | 10 | 10 | 2.8 |
| 12. | 90 | 23 | 10 | 5 | 3.1 |
| 13. | 45 | 23 | 10 | 10 | 2.9 |
| 14. | 45 | 23 | 10 | 5 | 3.0 |
| 15. | 23 | 23 | 10 | 10 | 2.9 |
| 16. | 23 | 23 | 10 | 5 | 2.9 |
| 17. | 23 | 45 | 11 | 25 | 3.0 |
| 18. | 23 | 45 | 10 | 10 | 3.0 |
| 19. | 9 | 23 | 10 | 10 | 3.1 |
| 20. | 11 | 23 | 10 | 25 | 3.1 |
| 21. | 9 | 23 | 10 | 5 | 2.8 |
| 22. | 5 | 23 | 10 | 10 | 3.1 |
| 23. | 5 | 23 | 10 | 50 | 3.1 |
| 24. | 5 | 23 | 10 | 5 | 2.7 |
| 25. | 5 | 45 | 12 | 5 | 3.0 |
| 26. | 1 | 1 | 10 | 7 | 3.5 |
| 27. | 1 | 1 | 10 | 7 | 3.0 |
| 28. | 0.1 | 0.1 | 10 | 15 | 3.5 |
| 29. | 0.1 | 0.1 | 10 | 15 | 3.0 |

C) The Table that follows is a compilation of formulation examples according to the invention of the R-enantiomer of the compound of Example 7 and the active substance 2.1 in the form of base and cation. 100 mL of medicament formulation contains the following, in purified water or water for injections:

| | 1′ (base) | 2.1′ (cation) | BA-Cl | EDTA | pH |
|---|---|---|---|---|---|
| Example | (mg) | (mg) | (mg) | (mg) | (HCl) |
| 1. | 1000 | 1000 | — | 2 | 2.9 |
| 2. | 500 | 500 | 10 | 10 | 2.7 |
| 3. | 500 | 250 | 4 | 10 | 2.9 |
| 4. | 500 | 250 | 5 | 10 | 2.9 |
| 5. | 500 | 85 | 10 | 11 | 2.9 |
| 6. | 250 | 500 | 10 | 9 | 3.1 |
| 7. | 85 | 500 | 8 | 10 | 2.9 |
| 8. | 200 | 45 | 15 | — | 2.9 |
| 9. | 200 | 23 | 11 | 12 | 2.7 |
| 10. | 200 | 23 | 10 | 10 | 2.7 |
| 11. | 90 | 23 | 10 | 10 | 2.8 |
| 12. | 90 | 23 | 10 | 5 | 3.1 |
| 13. | 45 | 23 | 10 | 10 | 2.9 |
| 14. | 45 | 23 | 10 | 5 | 3.0 |
| 15. | 23 | 23 | 10 | 10 | 2.9 |
| 16. | 23 | 23 | 10 | 5 | 2.9 |
| 17. | 23 | 45 | 11 | 25 | 3.0 |
| 18. | 23 | 45 | 10 | 10 | 3.0 |
| 19. | 9 | 23 | 10 | 10 | 3.1 |
| 20. | 11 | 23 | 10 | 25 | 3.1 |
| 21. | 9 | 23 | 10 | 5 | 2.8 |
| 22. | 5 | 23 | 10 | 10 | 3.1 |
| 23. | 5 | 23 | 10 | 50 | 3.1 |
| 24. | 5 | 23 | 10 | 5 | 2.7 |
| 25. | 5 | 45 | 12 | 5 | 3.0 |
| 26. | 1 | 1 | 10 | 7 | 3.5 |
| 27. | 1 | 1 | 10 | 7 | 3.0 |
| 28. | 0.1 | 0.1 | 10 | 15 | 3.5 |
| 29. | 0.1 | 0.1 | 10 | 15 | 3.0 |

D) The Table that follows is a compilation of formulation examples according to the invention of the R-enantiomer of the compound of Example 9 and the active substance 2.1 in the form of base and cation. 100 mL of medicament formulation contains the following, in purified water or water for injections:

| | 1′ (base) | 2.1′ (cation) | BA-Cl | EDTA | pH |
|---|---|---|---|---|---|
| Example | (mg) | (mg) | (mg) | (mg) | (HCl) |
| 1. | 1000 | 1000 | — | 2 | 2.9 |
| 2. | 500 | 500 | 10 | 10 | 2.7 |
| 3. | 500 | 250 | 4 | 10 | 2.9 |
| 4. | 500 | 250 | 5 | 10 | 2.9 |
| 5. | 500 | 85 | 10 | 11 | 2.9 |
| 6. | 250 | 500 | 10 | 9 | 3.1 |
| 7. | 85 | 500 | 8 | 10 | 2.9 |
| 8. | 200 | 45 | 15 | — | 2.9 |
| 9. | 200 | 23 | 11 | 12 | 2.7 |
| 10. | 200 | 23 | 10 | 10 | 2.7 |
| 11. | 90 | 23 | 10 | 10 | 2.8 |
| 12. | 90 | 23 | 10 | 5 | 3.1 |
| 13. | 45 | 23 | 10 | 10 | 2.9 |
| 14. | 45 | 23 | 10 | 5 | 3.0 |
| 15. | 23 | 23 | 10 | 10 | 2.9 |
| 16. | 23 | 23 | 10 | 5 | 2.9 |
| 17. | 23 | 45 | 11 | 25 | 3.0 |
| 18. | 23 | 45 | 10 | 10 | 3.0 |
| 19. | 9 | 23 | 10 | 10 | 3.1 |
| 20. | 11 | 23 | 10 | 25 | 3.1 |
| 21. | 9 | 23 | 10 | 5 | 2.8 |
| 22. | 5 | 23 | 10 | 10 | 3.1 |
| 23. | 5 | 23 | 10 | 50 | 3.1 |
| 24. | 5 | 23 | 10 | 5 | 2.7 |
| 25. | 5 | 45 | 12 | 5 | 3.0 |
| 26. | 1 | 1 | 10 | 7 | 3.5 |
| 27. | 1 | 1 | 10 | 7 | 3.0 |
| 28. | 0.1 | 0.1 | 10 | 15 | 3.5 |
| 29. | 0.1 | 0.1 | 10 | 15 | 3.0 |

E) The Table that follows is a compilation of formulation examples according to the invention of the R-enantiomer of the compound of Example 14 and the active substance 2.1 in the form of base and cation. 100 mL of medicament formulation contains the following, in purified water or water for injections:

| | 1′ (base) | 2.1′ (cation) | BA-Cl | EDTA | pH |
|---|---|---|---|---|---|
| Example | (mg) | (mg) | (mg) | (mg) | (HCl) |
| 1. | 1000 | 1000 | — | 2 | 2.9 |
| 2. | 500 | 500 | 10 | 10 | 2.7 |
| 3. | 500 | 250 | 4 | 10 | 2.9 |
| 4. | 500 | 250 | 5 | 10 | 2.9 |
| 5. | 500 | 85 | 10 | 11 | 2.9 |
| 6. | 250 | 500 | 10 | 9 | 3.1 |
| 7. | 85 | 500 | 8 | 10 | 2.9 |
| 8. | 200 | 45 | 15 | — | 2.9 |
| 9. | 200 | 23 | 11 | 12 | 2.7 |
| 10. | 200 | 23 | 10 | 10 | 2.7 |
| 11. | 90 | 23 | 10 | 10 | 2.8 |
| 12. | 90 | 23 | 10 | 5 | 3.1 |
| 13. | 45 | 23 | 10 | 10 | 2.9 |
| 14. | 45 | 23 | 10 | 5 | 3.0 |
| 15. | 23 | 23 | 10 | 10 | 2.9 |
| 16. | 23 | 23 | 10 | 5 | 2.9 |
| 17. | 23 | 45 | 11 | 25 | 3.0 |
| 18. | 23 | 45 | 10 | 10 | 3.0 |
| 19. | 9 | 23 | 10 | 10 | 3.1 |
| 20. | 11 | 23 | 10 | 25 | 3.1 |
| 21. | 9 | 23 | 10 | 5 | 2.8 |
| 22. | 5 | 23 | 10 | 10 | 3.1 |
| 23. | 5 | 23 | 10 | 50 | 3.1 |
| 24. | 5 | 23 | 10 | 5 | 2.7 |
| 25. | 5 | 45 | 12 | 5 | 3.0 |
| 26. | 1 | 1 | 10 | 7 | 3.5 |
| 27. | 1 | 1 | 10 | 7 | 3.0 |
| 28. | 0.1 | 0.1 | 10 | 15 | 3.5 |
| 29. | 0.1 | 0.1 | 10 | 15 | 3.0 |

F) The Table that follows is a compilation of formulation examples according to the invention of the R-enantiomer of the compound of Example 17 and the active substance 2.1 in the form of base and cation. 100 mL of medicament formulation contains the following, in purified water or water for injections:

| | 1′ (base) | 2.1′ (cation) | BA-Cl | EDTA | pH |
|---|---|---|---|---|---|
| Example | (mg) | (mg) | (mg) | (mg) | (HCl) |
| 1. | 1000 | 1000 | — | 2 | 2.9 |
| 2. | 500 | 500 | 10 | 10 | 2.7 |
| 3. | 500 | 250 | 4 | 10 | 2.9 |
| 4. | 500 | 250 | 5 | 10 | 2.9 |
| 5. | 500 | 85 | 10 | 11 | 2.9 |
| 6. | 250 | 500 | 10 | 9 | 3.1 |
| 7. | 85 | 500 | 8 | 10 | 2.9 |
| 8. | 200 | 45 | 15 | — | 2.9 |
| 9. | 200 | 23 | 11 | 12 | 2.7 |
| 10. | 200 | 23 | 10 | 10 | 2.7 |
| 11. | 90 | 23 | 10 | 10 | 2.8 |
| 12. | 90 | 23 | 10 | 5 | 3.1 |
| 13. | 45 | 23 | 10 | 10 | 2.9 |
| 14. | 45 | 23 | 10 | 5 | 3.0 |
| 15. | 23 | 23 | 10 | 10 | 2.9 |
| 16. | 23 | 23 | 10 | 5 | 2.9 |
| 17. | 23 | 45 | 11 | 25 | 3.0 |
| 18. | 23 | 45 | 10 | 10 | 3.0 |
| 19. | 9 | 23 | 10 | 10 | 3.1 |
| 20. | 11 | 23 | 10 | 25 | 3.1 |
| 21. | 9 | 23 | 10 | 5 | 2.8 |
| 22. | 5 | 23 | 10 | 10 | 3.1 |
| 23. | 5 | 23 | 10 | 50 | 3.1 |
| 24. | 5 | 23 | 10 | 5 | 2.7 |
| 25. | 5 | 45 | 12 | 5 | 3.0 |
| 26. | 1 | 1 | 10 | 7 | 3.5 |
| 27. | 1 | 1 | 10 | 7 | 3.0 |
| 28. | 0.1 | 0.1 | 10 | 15 | 3.5 |
| 29. | 0.1 | 0.1 | 10 | 15 | 3.0 |

G) The Table that follows is a compilation of formulation examples according to the invention of the R-enantiomer of the compound of Example 1 and the active substance 2.1 in the form of base and cation. 100 mL of medicament formulation contains:

| | | 2.1′ | EtOH/ | α- | | | | |
|---|---|---|---|---|---|---|---|---|
| | 1′ (base) | (cation) | water | tocopherol | BHT | BA-Cl | EDTA | pH |
| Example | (mg) | (mg) | (% V/V) | (mg) | (mg) | (mg) | (mg) | (HCl) |
| 1. | 10 | 10 | 20/80 | — | — | 10 | 10 | 2.7 |
| 2. | 9 | 23 | 20/80 | — | — | 10 | 10 | 2.9 |
| 3. | 90 | 23 | 50/50 | — | — | 5 | 1 | 2.7 |
| 4. | 23 | 23 | 50/50 | — | — | 10 | 2 | 2.9 |
| 5. | 10 | 10 | 50/50 | — | — | 10 | 3 | 2.9 |
| 6. | 5 | 23 | 50/50 | — | — | 5 | 4 | 3.1 |
| 7. | 1000 | 1000 | 70/30 | 50 | — | — | — | 3.0 |
| 8. | 500 | 250 | 70/30 | — | — | 5 | 0.5 | 3.0 |
| 9. | 85 | 500 | 70/30 | — | 100 | — | — | 2.7 |
| 10. | 90 | 23 | 70/30 | — | 100 | — | — | 3.1 |
| 11. | 45 | 23 | 70/30 | 50 | — | — | — | 3.3 |
| 12. | 23 | 23 | 70/30 | — | — | 5 | 0.5 | 2.7 |
| 13. | 9 | 23 | 70/30 | — | — | — | 1 | 2.9 |
| 14. | 5 | 23 | 70/30 | 50 | — | — | 2 | 3.1 |
| 15. | 23 | 45 | 70/30 | — | 100 | — | 3 | 3.3 |
| 16. | 200 | 23 | 70/30 | — | — | 5 | — | 3 |
| 17. | 90 | 23 | 70/30 | — | — | — | 2 | 4 |
| 18. | 23 | 23 | 70/30 | — | — | — | 2 | 5 |
| 19. | 5 | 23 | 70/30 | — | — | — | 2 | 3 |
| 20. | 1.0 | 45 | 70/30 | — | — | — | 1 | 3 |
| 21. | 500 | 500 | 80/20 | — | — | 5 | 1 | 3.0 |
| 22. | 500 | 250 | 80/20 | — | — | 3 | 0.5 | 2.8 |
| 23. | 85 | 500 | 80/20 | — | 100 | — | — | 3.2 |
| 24. | 5 | 23 | 80/20 | 50 | — | — | 0.5 | 3.5 |
| 25. | 1000 | 1000 | 90/10 | — | — | — | — | 5.0 |
| 26. | 1000 | 1000 | 90/10 | 50 | — | 5 | — | 3.0 |
| 27. | 500 | 250 | 90/10 | — | — | — | 0.5 | 3.0 |
| 28. | 500 | 250 | 90/10 | — | — | 5 | 0.5 | 3.0 |
| 29. | 85 | 500 | 90/10 | — | 100 | 5 | — | 2.7 |
| 30. | 90 | 23 | 90/10 | 50 | — | — | — | 3.0 |
| 31. | 45 | 23 | 90/10 | — | 100 | — | — | 3.0 |
| 32. | 23 | 45 | 90/10 | — | — | 10 | 0.5 | 2.9 |
| 33. | 23 | 23 | 90/10 | — | — | 5 | 1 | 3.1 |
| 34. | 9 | 23 | 90/10 | 50 | — | — | 1 | 3.5 |
| 35. | 5 | 23 | 90/10 | — | 100 | — | 1 | 3.5 |
| 36. | 0.5 | 45 | 90/10 | — | — | — | 1 | 4.0 |
| 37. | 1 | 1 | 95/5 | 50 | — | — | — | 3.0 |
| 38. | 0.1 | 0.1 | 95/5 | — | 100 | — | 0.5 | 3.5 |
| 39. | 23 | 23 | 95/5 | 50 | — | — | — | 2.7 |
| 40. | 45 | 45 | 95/5 | — | — | 5 | 0.5 | 3.0 |
| 41. | 85 | 500 | 95/5 | — | — | — | — | 3 |
| 42. | 2.5 | 1 | 95/5 | — | — | — | — | 4 |
| 43. | 0.5 | 3 | 95/5 | — | — | 5 | — | 5 |
| 44. | 10 | 10 | 100/0 | — | — | — | — | 3.0 |
| 45. | 10 | 10 | 100/0 | — | — | 5 | — | 4.0 |

H) The Table that follows is a compilation of formulation examples according to the invention of the R-enantiomer of the compound of Example 3 and the active substance 2.1 in the form of base and cation. 100 mil of medicament formulation contains:

| | | 2.1′ | EtOH/ | α- | | | | |
|---|---|---|---|---|---|---|---|---|
| | 1′ (base) | (cation) | water | tocopherol | BHT | BA-Cl | EDTA | pH |
| Example | (mg) | (mg) | (% V/V) | (mg) | (mg) | (mg) | (mg) | (HCl) |
| 1. | 10 | 10 | 20/80 | — | — | 10 | 10 | 2.7 |
| 2. | 9 | 23 | 20/80 | — | — | 10 | 10 | 2.9 |
| 3. | 90 | 23 | 50/50 | — | — | 5 | 1 | 2.7 |
| 4. | 23 | 23 | 50/50 | — | — | 10 | 2 | 2.9 |
| 5. | 10 | 10 | 50/50 | — | — | 10 | 3 | 2.9 |
| 6. | 5 | 23 | 50/50 | — | — | 5 | 4 | 3.1 |
| 7. | 1000 | 1000 | 70/30 | 50 | — | — | — | 3.0 |
| 8. | 500 | 250 | 70/30 | — | — | 5 | 0.5 | 3.0 |
| 9. | 85 | 500 | 70/30 | — | 100 | — | — | 2.7 |
| 10. | 90 | 23 | 70/30 | — | 100 | — | — | 3.1 |
| 11. | 45 | 23 | 70/30 | 50 | — | — | — | 3.3 |
| 12. | 23 | 23 | 70/30 | — | — | 5 | 0.5 | 2.7 |
| 13. | 9 | 23 | 70/30 | — | — | — | 1 | 2.9 |
| 14. | 5 | 23 | 70/30 | 50 | — | — | 2 | 3.1 |
| 15. | 23 | 45 | 70/30 | — | 100 | — | 3 | 3.3 |
| 16. | 200 | 23 | 70/30 | — | — | 5 | — | 3 |
| 17. | 90 | 23 | 70/30 | — | — | — | 2 | 4 |
| 18. | 23 | 23 | 70/30 | — | — | — | 2 | 5 |
| 19. | 5 | 23 | 70/30 | — | — | — | 2 | 3 |
| 20. | 1.0 | 45 | 70/30 | — | — | — | 1 | 3 |
| 21. | 500 | 500 | 80/20 | — | — | 5 | 1 | 3.0 |
| 22. | 500 | 250 | 80/20 | — | — | 3 | 0.5 | 2.8 |
| 23. | 85 | 500 | 80/20 | — | 100 | — | — | 3.2 |
| 24. | 5 | 23 | 80/20 | 50 | — | — | 0.5 | 3.5 |
| 25. | 1000 | 1000 | 90/10 | — | — | — | — | 5.0 |
| 26. | 1000 | 1000 | 90/10 | 50 | — | 5 | — | 3.0 |
| 27. | 500 | 250 | 90/10 | — | — | — | 0.5 | 3.0 |
| 28. | 500 | 250 | 90/10 | — | — | 5 | 0.5 | 3.0 |
| 29. | 85 | 500 | 90/10 | — | 100 | 5 | — | 2.7 |
| 30. | 90 | 23 | 90/10 | 50 | — | — | — | 3.0 |
| 31. | 45 | 23 | 90/10 | — | 100 | — | — | 3.0 |
| 32. | 23 | 45 | 90/10 | — | — | 10 | 0.5 | 2.9 |
| 33. | 23 | 23 | 90/10 | — | — | 5 | 1 | 3.1 |
| 34. | 9 | 23 | 90/10 | 50 | — | — | 1 | 3.5 |
| 35. | 5 | 23 | 90/10 | — | 100 | — | 1 | 3.5 |
| 36. | 0.5 | 45 | 90/10 | — | — | — | 1 | 4.0 |
| 37. | 1 | 1 | 95/5 | 50 | — | — | — | 3.0 |
| 38. | 0.1 | 0.1 | 95/5 | — | 100 | — | 0.5 | 3.5 |
| 39. | 23 | 23 | 95/5 | 50 | — | — | — | 2.7 |
| 40. | 45 | 45 | 95/5 | — | — | 5 | 0.5 | 3.0 |
| 41. | 85 | 500 | 95/5 | — | — | — | — | 3 |
| 42. | 2.5 | 1 | 95/5 | — | — | — | — | 4 |
| 43. | 0.5 | 3 | 95/5 | — | — | 5 | — | 5 |
| 44. | 10 | 10 | 100/0 | — | — | — | — | 3.0 |
| 45. | 10 | 10 | 100/0 | — | — | 5 | — | 4.0 |

I) The Table that follows is a compilation of formulation examples according to the invention of the R-enantiomer of the compound of Example 17 and the active substance 2.1 in the form of base and cation. 100 mL of medicament formulation contains:

| | | 2.1′ | EtOH/ | α- | | | | |
|---|---|---|---|---|---|---|---|---|
| | 1′ (base) | (cation) | water | tocopherol | BHT | BA-Cl | EDTA | pH |
| Example | (mg) | (mg) | (% V/V) | (mg) | (mg) | (mg) | (mg) | (HCl) |
| 1. | 10 | 10 | 20/80 | — | — | 10 | 10 | 2.7 |
| 2. | 9 | 23 | 20/80 | — | — | 10 | 10 | 2.9 |
| 3. | 90 | 23 | 50/50 | — | — | 5 | 1 | 2.7 |
| 4. | 23 | 23 | 50/50 | — | — | 10 | 2 | 2.9 |
| 5. | 10 | 10 | 50/50 | — | — | 10 | 3 | 2.9 |
| 6. | 5 | 23 | 50/50 | — | — | 5 | 4 | 3.1 |
| 7. | 1000 | 1000 | 70/30 | 50 | — | — | — | 3.0 |
| 8. | 500 | 250 | 70/30 | — | — | 5 | 0.5 | 3.0 |
| 9. | 85 | 500 | 70/30 | — | 100 | — | — | 2.7 |
| 10. | 90 | 23 | 70/30 | — | 100 | — | — | 3.1 |
| 11. | 45 | 23 | 70/30 | 50 | — | — | — | 3.3 |
| 12. | 23 | 23 | 70/30 | — | — | 5 | 0.5 | 2.7 |
| 13. | 9 | 23 | 70/30 | — | — | — | 1 | 2.9 |
| 14. | 5 | 23 | 70/30 | 50 | — | — | 2 | 3.1 |
| 15. | 23 | 45 | 70/30 | — | 100 | — | 3 | 3.3 |
| 16. | 200 | 23 | 70/30 | — | — | 5 | — | 3 |
| 17. | 90 | 23 | 70/30 | — | — | — | 2 | 4 |
| 18. | 23 | 23 | 70/30 | — | — | — | 2 | 5 |
| 19. | 5 | 23 | 70/30 | — | — | — | 2 | 3 |
| 20. | 1.0 | 45 | 70/30 | — | — | — | 1 | 3 |
| 21. | 500 | 500 | 80/20 | — | — | 5 | 1 | 3.0 |
| 22. | 500 | 250 | 80/20 | — | — | 3 | 0.5 | 2.8 |
| 23. | 85 | 500 | 80/20 | — | 100 | — | — | 3.2 |
| 24. | 5 | 23 | 80/20 | 50 | — | — | 0.5 | 3.5 |
| 25. | 1000 | 1000 | 90/10 | — | — | — | — | 5.0 |
| 26. | 1000 | 1000 | 90/10 | 50 | — | 5 | — | 3.0 |
| 27. | 500 | 250 | 90/10 | — | — | — | 0.5 | 3.0 |
| 28. | 500 | 250 | 90/10 | — | — | 5 | 0.5 | 3.0 |
| 29. | 85 | 500 | 90/10 | — | 100 | 5 | — | 2.7 |
| 30. | 90 | 23 | 90/10 | 50 | — | — | — | 3.0 |
| 31. | 45 | 23 | 90/10 | — | 100 | — | — | 3.0 |
| 32. | 23 | 45 | 90/10 | — | — | 10 | 0.5 | 2.9 |
| 33. | 23 | 23 | 90/10 | — | — | 5 | 1 | 3.1 |
| 34. | 9 | 23 | 90/10 | 50 | — | — | 1 | 3.5 |
| 35. | 5 | 23 | 90/10 | — | 100 | — | 1 | 3.5 |
| 36. | 0.5 | 45 | 90/10 | — | — | — | 1 | 4.0 |
| 37. | 1 | 1 | 95/5 | 50 | — | — | — | 3.0 |
| 38. | 0.1 | 0.1 | 95/5 | — | 100 | — | 0.5 | 3.5 |
| 39. | 23 | 23 | 95/5 | 50 | — | — | — | 2.7 |
| 40. | 45 | 45 | 95/5 | — | — | 5 | 0.5 | 3.0 |
| 41. | 85 | 500 | 95/5 | — | — | — | — | 3 |
| 42. | 2.5 | 1 | 95/5 | — | — | — | — | 4 |
| 43. | 0.5 | 3 | 95/5 | — | — | 5 | — | 5 |
| 44. | 10 | 10 | 100/0 | — | — | — | — | 3.0 |
| 45. | 10 | 10 | 100/0 | — | — | 5 | — | 4.0 |

J) The Table that follows is a compilation of formulation examples according to the invention of the R-enantiomer of the compound of Example 13 and the active substance 2.1 in the form of base and cation. 100 mL of medicament formulation contains:

| | | 2.1′ | EtOH/ | α- | | | | |
|---|---|---|---|---|---|---|---|---|
| | 1′ (base) | (cation) | water | tocopherol | BHT | BA-Cl | EDTA | pH |
| Example | (mg) | (mg) | (% V/V) | (mg) | (mg) | (mg) | (mg) | (HCl) |
| 1. | 10 | 10 | 20/80 | — | — | 10 | 10 | 2.7 |
| 2. | 9 | 23 | 20/80 | — | — | 10 | 10 | 2.9 |
| 3. | 90 | 23 | 50/50 | — | — | 5 | 1 | 2.7 |
| 4. | 23 | 23 | 50/50 | — | — | 10 | 2 | 2.9 |
| 5. | 10 | 10 | 50/50 | — | — | 10 | 3 | 2.9 |
| 6. | 5 | 23 | 50/50 | — | — | 5 | 4 | 3.1 |
| 7. | 1000 | 1000 | 70/30 | 50 | — | — | — | 3.0 |
| 8. | 500 | 250 | 70/30 | — | — | 5 | 0.5 | 3.0 |
| 9. | 85 | 500 | 70/30 | — | 100 | — | — | 2.7 |
| 10. | 90 | 23 | 70/30 | — | 100 | — | — | 3.1 |
| 11. | 45 | 23 | 70/30 | 50 | — | — | — | 3.3 |
| 12. | 23 | 23 | 70/30 | — | — | 5 | 0.5 | 2.7 |
| 13. | 9 | 23 | 70/30 | — | — | — | 1 | 2.9 |
| 14. | 5 | 23 | 70/30 | 50 | — | — | 2 | 3.1 |
| 15. | 23 | 45 | 70/30 | — | 100 | — | 3 | 3.3 |
| 16. | 200 | 23 | 70/30 | — | — | 5 | — | 3 |
| 17. | 90 | 23 | 70/30 | — | — | — | 2 | 4 |
| 18. | 23 | 23 | 70/30 | — | — | — | 2 | 5 |
| 19. | 5 | 23 | 70/30 | — | — | — | 2 | 3 |
| 20. | 1.0 | 45 | 70/30 | — | — | — | 1 | 3 |
| 21. | 500 | 500 | 80/20 | — | — | 5 | 1 | 3.0 |
| 22. | 500 | 250 | 80/20 | — | — | 3 | 0.5 | 2.8 |
| 23. | 85 | 500 | 80/20 | — | 100 | — | — | 3.2 |
| 24. | 5 | 23 | 80/20 | 50 | — | — | 0.5 | 3.5 |
| 25. | 1000 | 1000 | 90/10 | — | — | — | — | 5.0 |
| 26. | 1000 | 1000 | 90/10 | 50 | — | 5 | — | 3.0 |
| 27. | 500 | 250 | 90/10 | — | — | — | 0.5 | 3.0 |
| 28. | 500 | 250 | 90/10 | — | — | 5 | 0.5 | 3.0 |
| 29. | 85 | 500 | 90/10 | — | 100 | 5 | — | 2.7 |
| 30. | 90 | 23 | 90/10 | 50 | — | — | — | 3.0 |
| 31. | 45 | 23 | 90/10 | — | 100 | — | — | 3.0 |
| 32. | 23 | 45 | 90/10 | — | — | 10 | 0.5 | 2.9 |
| 33. | 23 | 23 | 90/10 | — | — | 5 | 1 | 3.1 |
| 34. | 9 | 23 | 90/10 | 50 | — | — | 1 | 3.5 |
| 35. | 5 | 23 | 90/10 | — | 100 | — | 1 | 3.5 |
| 36. | 0.5 | 45 | 90/10 | — | — | — | 1 | 4.0 |
| 37. | 1 | 1 | 95/5 | 50 | — | — | — | 3.0 |
| 38. | 0.1 | 0.1 | 95/5 | — | 100 | — | 0.5 | 3.5 |
| 39. | 23 | 23 | 95/5 | 50 | — | — | — | 2.7 |
| 40. | 45 | 45 | 95/5 | — | — | 5 | 0.5 | 3.0 |
| 41. | 85 | 500 | 95/5 | — | — | — | — | 3 |
| 42. | 2.5 | 1 | 95/5 | — | — | — | — | 4 |
| 43. | 0.5 | 3 | 95/5 | — | — | 5 | — | 5 |
| 44. | 10 | 10 | 100/0 | — | — | — | — | 3.0 |
| 45. | 10 | 10 | 100/0 | — | — | 5 | — | 4.0 |

We claim:

1. A pharmaceutical formulation, comprising:
(a) a compound of formula 1 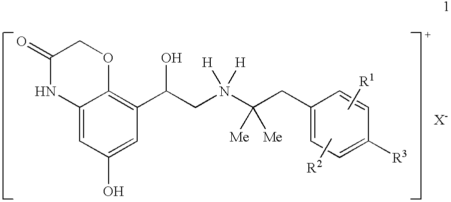
wherein:
R¹ is hydrogen, C₁₋₄-alkyl, —O—C₁₋₄-alkyl, or halogen;
R² is hydrogen, C₁₋₄-alkyl, —O—C₁₋₄-alkyl, or halogen;
R³ is hydrogen, C₁₋₄-alkyl, —O—C₁₋₄-alkyl, halogen, OH, —O—C₁₋₄-alkylene-COOH, or —O—C₁₋₄-alkylene-COO—C₁₋₄-alkyl; and
X⁻ denotes an anion with a single negative charge, or a tautomer, enantiomer, solvate, or hydrate thereof;
(b) a second active substance 2 selected from tiotropium salts, oxitropium salts, flutropium salts, ipratropium salts, glycopyrronium salts, and trospium salts, or a tautomer, enantiomer, solvate, or hydrate thereof;
(c) at least one pharmacologically acceptable acid; and
(d) a solvent selected from water, ethanol, or a mixture of water and ethanol.

2. The pharmaceutical formulation according to claim 1, wherein:
X⁻ is chloride, bromide, iodide, sulfate, phosphate, methanesulfonate, nitrate, maleate, acetate, benzoate, citrate, salicylate, trifluoroacetate, fumarate, tartrate, oxalate, succinate, benzoate, or p-toluenesulfonate.

3. The pharmaceutical formulation according to claim 1, wherein:
R¹ is hydrogen, methyl, ethyl, fluorine, or chlorine;
R² is hydrogen, methyl, ethyl, fluorine, or chlorine; and
R³ is hydrogen, methyl, ethyl, propyl, OH, methoxy, ethoxy, fluorine, chlorine, bromine, —O—CH₂—COOH, —O—CH₂—COOmethyl, —O—CH₂—COOethyl, —O—CH₂—CH₂COOH, —O—CH₂—CH₂COOmethyl, —O—CH₂—CH₂COOethyl, —O—CH₂—CH₂—CH₂COOH, —O—CH₂—CH₂—CH₂COOmethyl, or —O—CH₂—CH₂—CH₂COOethyl.

4. The pharmaceutical formulation according to claim 1, wherein:
R¹ is hydrogen or methyl, preferably hydrogen;
R² is hydrogen or methyl, preferably hydrogen;
R³ is methyl, OH, methoxy, fluorine, chlorine, bromine, —O—CH₂—COOH, or —O—CH₂-COOethyl; and
X⁻ is chloride, bromide, sulfate, methanesulfonate, maleate, acetate, benzoate, citrate, salicylate, trifluoroacetate, fumarate, tartrate, or succinate.

5. The pharmaceutical formulation according to one of claims 1 to 4, wherein the second active substance 2 is tiotropium bromide, oxitropium bromide, or ipratropium bromide, or a tautomer, enantiomer, solvate, or hydrate thereof.

6. The pharmaceutical formulation according to one of claims 1 to 4, wherein the pharmacologically acceptable acid is selected from the inorganic acids hydrochloric acid, hydrobromic acid, nitric acid, sulfuric acid and phosphoric acid or from the organic acids ascorbic acid, citric acid, malic acid, tartaric acid, maleic acid, succinic acid, fumaric acid, acetic acid, formic acid, propionic acid, sorbic acid, benzoic acid, methanesulfonic acid, or benzenesulfonic acid.

7. The pharmaceutical formulation according to one of claims 1 to 4, wherein the pH is 2.0 to 6.5.

8. The pharmaceutical formulation according to one of claims 1 to 4, further comprising benzalkonium chloride.

9. The pharmaceutical formulation according to claim 8, wherein the benzalkonium chloride concentration is 1 to 50 mg per 100 mL of the pharmaceutical formulation.

10. The pharmaceutical formulation according to one of claims 1 to 4, further comprising an antioxidant.

11. The pharmaceutical formulation according to claim 10, wherein the antioxidant is ascorbic acid, propylgallate, butylhydroxyanisol, butylhydroxytoluene, tert-butylhydroxyquinone, or a tocopherol.

12. The pharmaceutical formulation according to one of claims 1 to 4, further comprising a complexing agent.

13. The pharmaceutical formulation according to claim 12, wherein the complexing agent concentration is 0.1 to 50 mg per 100 mL of the pharmaceutical formulation.

14. The pharmaceutical formulation according to one of claims 1 to 4, wherein the solvent is water.

15. The pharmaceutical formulation according to one of claims 1 to 4, wherein the solvent is ethanol.

16. The pharmaceutical formulation according to one of claims 1 to 4, wherein the solvent is a mixture of water and ethanol.

17. The pharmaceutical formulation according to claim 16, wherein the percentage proportion of ethanol by mass in the solvent is 5% to 99% ethanol.

18. The pharmaceutical formulation according to claim 1, wherein the second active substance 2 is a tiotropium salt, or a tautomer, enantiomer, solvate, or hydrate thereof.

19. The pharmaceutical formulation according to claim 1, wherein the amount of the compound of formula 1 (considered as the free base 1′) and the second active substance tiotropium salt (2.1′) are each independently about 0.1 to 2000 mg per 100 mL of the pharmaceutical formulation.

20. A pharmaceutical formulation, comprising:
(a) a free base of formula 1′ 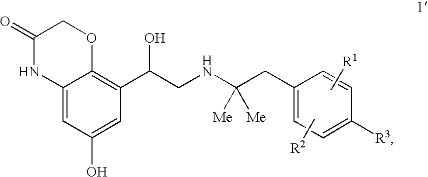
wherein:
R¹ is hydrogen, C₁₋₄-alkyl, —O—C₁₋₄-alkyl, or halogen;
R² is hydrogen, C₁₋₄-alkyl, —O—C₁₋₄-alkyl, or halogen; and
R³ is hydrogen, C₁₋₄-alkyl, —O—C₁₋₄-alkyl, halogen, OH, —O—C₁₋₄-alkylene-COOH, or —O—C₁₋₄-alkylene-COO—C₁₋₄-alkyl,
or a tautomer, enantiomer, solvate, or hydrate thereof;
(b) a second active substance 2 selected from tiotropium salts, oxitropium salts, flutropium salts, ipratropium salts, glycopyrronium salts, and trospium salts, or a tautomer, enantiomer, solvate, or hydrate thereof;
(c) at least one pharmacologically acceptable acid; and
(d) a solvent selected from water, ethanol, or a mixture of water and ethanol.
